(19) United States
(12) Patent Application Publication
(10) Pub. No.: US 2005/0112547 A1
(43) Pub. Date: May 26, 2005

(54) Vaginal infection test system and method

(75) Inventors: Ellis Quinn Youngkin, Boca Raton, FL (US); Pamela B. Lester, Boynton Beach, FL (US)

Correspondence Address: AKERMAN SENTERFITT, P.O. BOX 3188, WEST PALM BEACH, FL, 33402-3188, US

(73) Assignee: Florida Atlantic University, Boca Raton, FL (US)

(21) Appl. No.: 10/946,865

(22) Filed: Sep. 22, 2004

Related U.S. Application Data
(60) Provisional application No. 60/504,852, filed on Sep. 22, 2003.

(51) Int. Cl. C12Q 1/00

(52) U.S. Cl. 435/4

(57) ABSTRACT

Systems, methods and kits for detecting and diagnosing the presence of bacterial vaginosis (BV). Systems include a sample collector, a test chamber, a pH tester, an amine tester and instructions for performing and evaluating diagnostic tests predictive of BV. The systems and methods are suitable for use outside of a clinical setting.

### CROSS REFERENCE TO RELATED APPLICATIONS

This application claims the benefit of U.S. Provisional application Ser. No. 06/504,852, filed Sep. 22, 2003 and entitled “Vaginal Infection Test System and Method”, which is incorporated herein in its entirety.

### STATEMENT AS TO FEDERALLY SPONSORED RESEARCH

Not applicable.

### FIELD OF THE INVENTION

This invention relates generally to the fields of clinical medicine, medicine, nursing, infectious diseases and women's health issues. More particularly, it relates to a system, methods and kits for detecting and diagnosing vaginal infections such as bacterial vaginosis (BV).

### BACKGROUND

Normal healthy vaginal tissue is populated by protective vaginal bacterial flora, such as hydrogen peroxide-producing lactobacilli. Bacterial vaginosis (BV) is an infection involving an imbalance in the vaginal bacterial flora. In this condition, the lactobacilli and other normal flora have been reported to be replaced with pathogenic anaerobic coccobacilli. Studies associate BV with a host of serious conditions affecting the female reproductive system, encompassing complications of pregnancy, childbirth and fertility. These include preterm birth, low birth weight infants, premature rupture of the membranes, chorionic and amnionic infection, infertility, ectopic pregnancy, and other perinatal/reproductive tract complications. Research studies have shown that diagnosis and treatment of BV in pregnancy reduces rates of premature delivery. Recent data from clinical trials support reduced risk of preterm birth with early detection and treatment of BV in women with a prior history of preterm birth.

BV is common in women of childbearing age, occurring from 10% to 64% in low to high risk populations. Besides its deleterious effects in pregnant women, another serious consequence of BV is an increased susceptibility to the HIV virus and other sexually transmitted diseases (STDs), such as chlamydia and gonorrhea. One study states that a type of BV typified by inflammation, i.e., BV II, should be classified as an STD based on its resemblance to Chlamydia trachomatis infection, and concludes that the presence of BV may be predictive of the presence of other STDs. Unfortunately, BV is often asymptomatic in the early stages, and therefore can go undetected, and lead to serious complications when left unchecked.

### SUMMARY

The invention provides systems, methods and kits for predicting, detecting and diagnosing the presence of bacterial vaginosis (BV). A component of the systems and kits is a device that includes a sample collector for obtaining a biological sample from a subject, a test chamber, a pH tester for determining the pH of the biological sample, and an amine tester for determining the presence or absence of amines in the sample. Instructions are provided for assigning a numerical value to information derived from at least one determination made using the device, and for comparing the value to a predetermined control value of a non-BV infected subject. This permitsa detection to be made regarding the presence or absence of BV. The system can also include an optional scoring system for tabulating the values obtained, and, in another embodiment, a sample preservation unit.

According to the criteria of the Centers for Disease Control, a diagnosis of BV can be made upon a showing of at least three of four indicators, including: 1) the presence of a coaty white vaginal discharge, 2) vaginal pH greater than 4.5, 3) positive results upon KOH amine testing (“whiff test”), and 4) clue cells found via microscopy.

In a preferred embodiment, the invention provides a system for diagnosing the presence of bacterial vaginosis comprising a sample collector for obtaining a biological sample from a subject; a test chamber having an optically transparent portion to allow visual determination of an appearance of said sample while said sample collector is in said chamber; a pH tester to indicate a pH of said biological sample; and an amine tester to evolve vapors from said sample for determination of a presence or absence of amines in said sample. The system further comprises a scoring system for tabulating a value, wherein a tabulated score above a control value is diagnostic of bacterial vaginosis.

In another preferred embodiment, the system detects at least one indicator according to the criteria of the Centers for Disease Control, more preferably, the system detects at least two indicators according to the criteria of the Centers for Disease Control; more preferably, the system detects at least three indicators according to criteria of the Centers for Disease Control; more preferably, the system detects four indicators according to the criteria of the Centers for Disease Control.

In accordance with the invention, the system detects at least one of the indicators: 1) the presence of a coaty white vaginal discharge, 2) vaginal pH greater than 4.5, 3) positive results upon KOH amine testing (“whiff test”), and 4) clue cells found via microscopy. Preferably, the system detects at least two of the indicators: 1) the presence of a coaty white vaginal discharge, 2) vaginal pH greater than 4.5, 3) positive results upon KOH amine testing (“whiff test”), and 4) clue cells found via microscopy; more preferably the system detects at least three of the indicators: 1) the presence of a coaty white vaginal discharge, 2) vaginal pH greater than 4.5, 3) positive results upon KOH amine testing (“whiff test”), and 4) clue cells found via microscopy; more preferably, the system detects four of the indicators: 1) the presence of a coaty white vaginal discharge, 2) vaginal pH greater than 4.5, 3) positive results upon KOH amine testing (“whiff test”), and 4) clue cells found via microscopy.

In a preferred embodiment, the system detects any combination of a plurality of indicators according to the criteria of the Centers for Disease Control.

In a preferred embodiment, the invention provides a method for diagnosing the presence of BV includes the steps of: (a) providing a biological sample from a subject, (b) determining a value for at least one indicator of BV, and (c) determining a sum of the value(s). To predict the presence or absence of BV in the sample, the sum from step (c) is compared to a control sum derived from at least one indicator of BV from a vaginal sample from a normal subject. Indicators of BV can include one or more of the appearance of the sample, the pH of the sample, and the presence or absence of amines therein.

The systems, methods and kits of the invention are suitable for use by a non-medically trained person, such as a consumer, and are designed to alert the user to the existence of indicia of increased likelihood of a condition for which medical treatment should be sought.

In another preferred embodiment, the invention provides a system for detecting and diagnosing the presence of bacterial vaginosis comprising a sample collector for obtaining a biological sample from a subject; a test chamber having an optically transparent portion to allow visual determination of an appearance of said sample while said sample collector is in said chamber; a pH tester to indicate a pH of said biological sample; an amine tester to evolve vapors from said sample for determination of a presence or absence of amines in said sample; and a sample preservation unit. Preferably, the sample preservation unit further comprises a container dimensionally proportioned to accommodate a sample collector and a cap, wherein the cap forms a leak-proof seal when affixed to the sample preservation unit.

In another preferred embodiment, the sample preservation unit contains a sample preserving medium, wherein the sample collector comprising a biological sample contacts sample preservation medium contained in the sample preservation unit. Preferably, the biological sample comprises cells from human vaginal fluid.

In a preferred embodiment, the invention provides a kit for detecting the presence of bacterial vaginosis comprising a sample collector for obtaining a biological sample from a subject; a test chamber having an optically transparent portion to allow visual determination of an appearance of said sample while said sample collector is in said chamber; a pH tester to indicate a pH of said biological sample; an amine tester to evolve vapors from said sample for determination of a presence or absence of amines in said sample; and instructions for assigning a numerical value to at least one of said appearance, said pH and said presence or absence of amines, and comparing said value to a predetermined control value provided.

In a preferred embodiment, detection of the presence of BV using the kit, includes the steps of: (a) providing a biological sample from a subject, (b) determining a value for at least one indicator of BV, and (c) determining a sum of the value(s). To predict the presence or absence of BV in the sample, the sum from step (c) is compared to a control sum derived from at least one indicator of BV from a vaginal sample from a normal subject. Indicators of BV can include one or more of the appearance of the sample, the pH of the sample, and the presence or absence of amines therein.

In another preferred embodiment, the kit provides for the visual determination of sample. The visual determination indicative of bacterial vaginosis is correlated with a white or gray color and a smooth or frothy texture, and a non-bacterial vaginosis sample is correlated with a yellow, green, brown or red color and a clumpy texture.

In another preferred embodiment, the kit provides for a pH indicator of bacterial vaginosis includes a pH of said sample. Preferably, the bacterial vaginosis-predictive pH value is correlated with a pH of 5.0 or higher, and a bacterial vaginosis-non-predictive value is correlated with a pH of 4.5 or lower.

In another preferred embodiment, the kit provides for the detection of the presence or absence of bacterial vaginosis. Preferably, the bacterial vaginosis-predictive value is correlated with the presence of amines in said sample, and a bacterial vaginosis-non-predictive value is correlated with the absence of amines in said sample. The presence of amines in said sample are detectable by a fish-like odor in the presence of potassium hydroxide in a concentration of between about 10% to 20% v/v and/or the presence of amines in said sample are detected by an amine indicator. The amine indicator can include for example, bromocresol green, bromophenol blue, bromocresol purple, bromochlorophenol blue, or nitrazine yellow.

Other aspects of the invention are described infra.

### BRIEF DESCRIPTION OF THE DRAWINGS

A fuller understanding of the present invention and the features and benefits thereof will be accomplished upon review of the following detailed description together with the accompanying drawings, in which:

FIGS. 1A-1E is an illustration showing a system for detecting the presence or absence of BV, according to an embodiment of the invention. FIG. 1A is an illustration showing one embodiment of a system according to the invention. FIG. 1B is an illustration showing a pH determining system 250, such as a color-coded chart. FIG. 1C is an illustration showing an embodiment in which the pH paper is incorporated into the collection tip 218. FIG. 1D is an illustration showing an embodiment of a sample preservation system. FIG. 1E is an illustration showing a score card.

### DETAILED DESCRIPTION

The invention provides in one aspect a system for predicting, detecting, and diagnosing the presence of bacterial vaginosis (BV) in a subject. According to the criteria of the Centers for Disease Control, a diagnosis of BV can be made upon a showing of at least three of four indicators, including: 1) the presence of a coaty white vaginal discharge, 2) vaginal pH greater than 4.5, 3) positive results upon KOH amine testing (“whiff test”), and 4) clue cells found via microscopy.

Prior to setting forth the invention, it may be helpful to an understanding thereof to set forth definitions of certain terms that will be used hereinafter.

“Diagnostic” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

“Detect” refers to identifying the presence or absence of bacterial vaginosis. Detection of bacterial vaginosis is based on the criteria set forth by the Centers for Disease Control. According to the criteria of the Centers for Disease Control, a diagnosis of BV can be made upon a showing of at least three of four indicators, including: 1) the presence of a coaty white vaginal discharge, 2) vaginal pH greater than 4.5, 3) positive results upon KOH amine testing (“whiff test”), and 4) clue cells found via microscopy.

“Sample” is used herein in its broadest sense. A sample comprising cells, polynucleotides, polypeptides, peptides, antibodies and the like may comprise a bodily fluid; a soluble fraction of a cell preparation, or media in which cells were grown; a chromosome, an organelle, or membrane isolated or extracted from a cell; genomic DNA, RNA, or cDNA, polypeptides, or peptides in solution or bound to a substrate; a cell; a tissue; a tissue print; a fingerprint, skin or hair; and the like. In a preferred embodiment, the sample is obtained from vaginal fluid.

“Clinician” refers to any medically trained personnel, including physicians, nurse practitioners, nurse midwives and physician assistants.

Prior to the invention, no systems, methods or kits were available to consumers to allow them to predict the presence of BV using indicators 1-3 above. The kit includes a sample collector for obtaining a biological sample from the vagina of a subject, a test chamber, a pH tester to determine a pH of the biological sample, and an amine tester to determine the presence or absence of amines in the sample. Instructions for use are also provided.

The systems and kits are suitable for use in a clinical setting, but may also be conveniently used by a consumer or a non-medically trained person, to make a detection of the presence or absence of BV in the privacy and convenience of any setting of the user's choice, such as at home. The devices incorporate in a simple, convenient and compact unit, components for performing tests heretofor traditionally practiced in a clinical setting to diagnose BV.

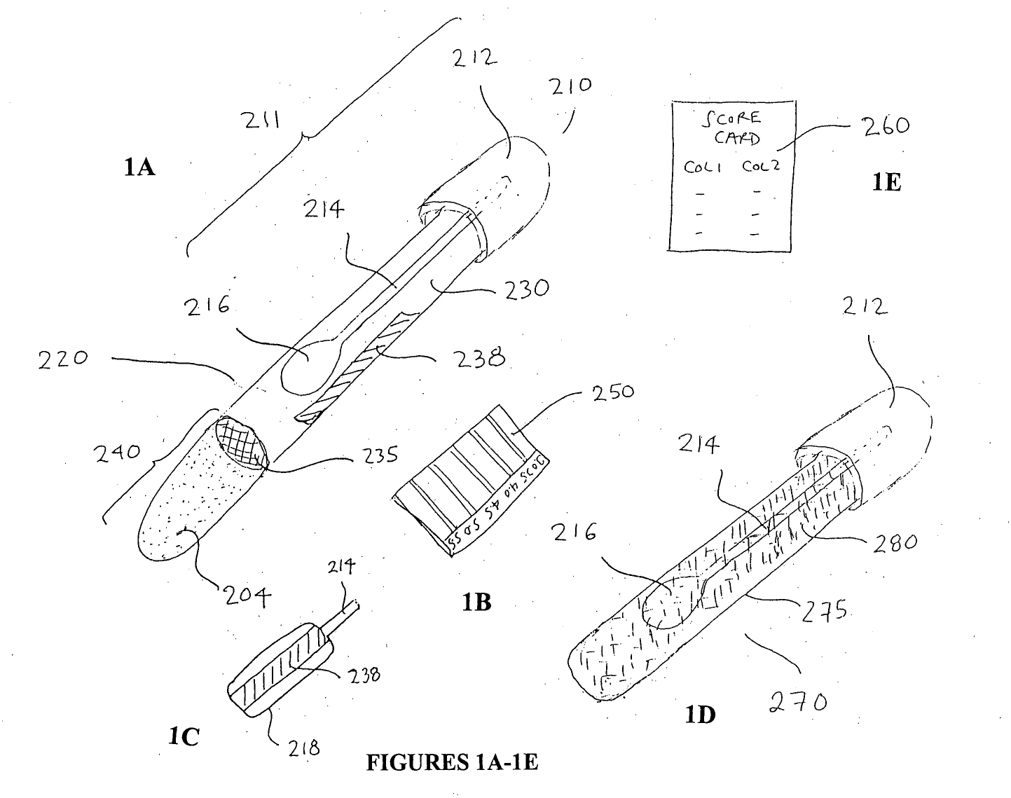

FIG. 1A shows one embodiment of a system according to the invention. The system (FIG. 1A) includes a medically aseptic device 210 including a removable sample collector 211 and a test chamber 220 adapted for use with the collector 211. Methods of sterilizing and packaging medically aseptic materials and related devices are well known to those having ordinary skill in the art. For example, methods disclosed in U.S. Pat. No. 4,973,449, which is incorporated herein by reference. The collector 211 can include a holder 212, a shaft 214 and a collection tip 216. The collection tip 216 is used to collect a specimen of vaginal fluid or discharge following insertion of the shaft 214 into the vagina while grasping the holder 212. Any material suitable for collection and visual inspection of vaginal fluids can be used for the collection tip 216. An exemplary material for the collection tip 216 is cotton gauze or fiber, preferably white in color, to allow for visual determination of any color present in the vaginal sample.

In the embodiment shown in FIG. 1B, the shaft 214 is affixed to the holder 212, and the holder 212 is in the form a cap attachable to the open end (not shown) of the test chamber 220 of the device 210. The handle 214 and the collection tip 216 of the collector 211 are preferably proportioned to fit inside the test chamber 220. The combined length of the shaft 214 and the collection tip 216 of the collector 211 can be any length suitable for collection of a sample from a vagina. The combined length is preferably in the range of 1-12 inches, and is more preferably in the range of 1-8 inches. In another preferred embodiment, the collection tip is between about 0.1 inches to about 5 inches in length.

In some embodiments (FIG. 1A), the holder 212 can create a leak-proof seal when affixed to the open end of the chamber 220. Any suitable arrangement for providing a leak-proof seal can be used. For example, the holder 212 can be attached to the test chamber 220 using threading (not shown) in the holder 212 that can screw into suitably positioned complementary threading (not shown) proximate to the open end of the test chamber 220. Alternatively, the holder 212 may snap in place, or may include an extension, for example a rubber stopper, that fits inside the test chamber 220 so as to form a leak-proof seal. In the embodiment of the device 210 shown in FIG. 1A, the test chamber 220 is divided into a proximal portion 230 and a distal portion 240, separated by a breakable seal 235. As further described below, in the operation of the device 210, the test chamber 220 can be used for several purposes in the operation of the device including: 1) evaluation of the sample appearance, 2) measurement of the sample pH, and 3) amine testing of the sample. Results of tests performed using the device 210 can provide the user with information needed to derive a numerical value, determined according to provided instructions, that enables a detection regarding the presence or absence of BV.

The test chamber 220 (FIG. 1A) allows for execution of desired test procedures. One of the test procedures can be visual inspection of the biological sample. To facilitate visual inspection and determination of appearance criteria of the vaginal sample, the proximal portion 230 of the test chamber 220 is preferably constructed of an optically transparent material, such as polystyrene or other plastic, or glass. Examples of other optically transparent materials include, but are not limited to plastics, quartz, silicon, polymers, gels, resins, carbon, membranes, glass, etc., or can include a combination of several types of materials such as a polymer blend, polymer coated glass and the like.

As previously described, the collection tip 216 (FIG. 1A) is preferably fabricated from a white colored material to aid visual inspection of the biological sample obtained from the subject.

A second test procedure for prediction of BV can be analysis of pH of the biological sample. The embodiment of the device 210 shown in FIG. 1A is equipped with a pH tester 238, situated on the inside of the test chamber 220. This position allows the pH tester 238 to be contacted with the vaginal fluid sample (not shown) present on the collection tip 216 following collection of the vaginal specimen and insertion of the collector 211 into the test chamber 220. The pH tester can be adhered to the inside of the test chamber 220 by an adhesive which may be any commercially available adhesive suitable for adhering such materials and would include, for example, an acrylic, pressure-sensitive two sided adhesive tape such as those manufactured by 3M Company Canada Inc. Any suitable pH tester can be used with the invention. In the illustrated embodiment shown as the device 210, the pH tester is pH test paper, fabricated in a strip. Any suitable size or shape of pH test paper can be used.

In another preferred embodiment, the invention provides analysis of a biological sample for a pH equal to or greater than a critical point in the range of 4.6 to 4.8 (preferably about 4.7) by a detectable transition, in combination with a positive control that shows a similarly detectable transition at a lower pH, the two changes being independently visible on the test device 210 (FIG. 1A). The positive control is useful in assuring that the indicator is not malfunctioning for reasons such as manufacturing error in the device, and that the device has been exposed to sufficient sample to produce a reading if the sample were indeed positive. In preferred embodiments, the test device also contains a negative control, independently visible, that includes an indicator whose change is caused not by application of a sample but by the decomposition or deterioration of the indicator itself. This device is of particular utility in the diagnosis of bacterial vaginosis as it can be used to test for three of the four tests in the Amsel criteria for bacterial vaginosis.

As explained in detail below, one implementation of the concepts of this invention is a combination test device for BV in vaginal fluid that shows a sharp visual transition at pH 4.7, and provides a reliable smell and/or visual indication of the presence of amines indicative of BV. For example, pH indicators can be used. An example, nitrazine yellow which, when in combination with quaternary ammonium groups, changes directly from greenish-yellow to blue over a narrow pH range of approximately 0.1 pH units as the pH rises, the transition centering around pH 4.7. The independently visible positive control changes color at a lower pH encountered when any specimen of the fluid is applied, regardless of whether or not BV is present. The two transitions are independently visible such that the occurrence of both indicates a pH of 4.7 or higher, while the occurrence of only the lower-pH transition (the positive control) indicates a pH below 4.7. The two transitions can be arranged in any pattern if desired. For example, a geometric pattern that serves as an indication of the result wherein a pair of orthogonal bars form a plus sign, the horizontal bar containing the indicator that changes color at low pH and the vertical bar containing the nitrazine yellow and quaternary ammonium groups. A specimen with a pH below 4.7 thereby produces a minus sign by creating a color change in the horizontal bar only, while a specimen with a pH above 4.7 produces a plus sign by creating color changes in both horizontal and vertical bars. The background area serves as the negative control. The test method is particularly suited for the detection of BV, and so, in a preferred embodiment, the sample is taken as a vaginal swab or sample from a female human patient. It should be noted that the present invention is useful in a wide range of applications in which diagnostic or other tests are to be performed upon a bodily discharge. By way of a non-limiting but preferred example, the invention will be described in the context of sampling vaginal secretions. It will be self-evident to one ordinarily skilled in the art that the invention may readily be applied also to pads, bandages, diapers and other absorbent elements for generating biological samples from regular or irregular bodily discharges of substantially any type, and for use in substantially any test.

In other embodiments of the invention, the pH tester 238 can be incorporated into or otherwise included in the collection tip of the collector 211. For example, as shown in FIG. 1C, the pH tester can be a strip of pH paper that is adherent to a portion of the collection tip. In the embodiment shown, the collection tip 218 (FIG. 1C) is generally larger than the tip 216 illustrated in FIG. 1A. However, the collection tip is dimensionally proportioned to prevent discomfort in a female subject when inserted into a vagina for collection of sample. The collection tip can be manufactured in a broad range of sizes. For example, the collection tip 218 can be about the size of a typical ear bud up to about the size of a typical vaginal tampon, and the pH tester can cover a portion of the surface of the tip 218. In the operation of embodiments incorporating the pH tester 238 into the collection tip 218, the steps of sample collection and pH testing are accomplished simultaneously upon contact of the collection tip 218 with the biological sample from the subject.

In embodiments of the device 210 utilizing pH paper as the pH tester, the device 210 can include a pH determining system 250, such as the color-coded chart 250 illustrated in FIG. 1B, which facilitates correlating the color of the pH paper with a known pH value. For example, the pH determining system 250 can be in the form of a chart including bars of color ranging in shades of yellow to green, correlated with corresponding indicators of pH values, for example from 3.0 to 7.0, in increments of, for example, 0.5 pH units. pH testing papers and the corresponding color-coding charts for determining pH are well known in the art. Any suitable pH increments can be used, as can any pH paper that provides, upon wetting with a sample, observable changes in color corresponding to pH values in the desired pH range (i.e., pH 2.0-10.0, and preferably pH 3.0-7.0).

The pH determining system 250 can be a separate component of the device 210, as illustrated in FIG. 1B, or it can be incorporated into the test chamber 220, for example by adhering it to a wall of the test chamber 220 (FIG. 1A). In this embodiment, the pH determining system 250 is preferably positioned proximate to the location of the pH paper on the wall of the test chamber 220 (FIG. 1A), to facilitate comparison of the color of the pH tester 238 with the colors on the pH determining system 250 (FIG. 1B). In other embodiments in which the pH determining system 250 (FIG. 1B) is a separate component not incorporated into the device 210 (FIG. 1A), pH can determined by holding the pH determining system 250 against the wall of test chamber 230 (FIG. 1A) close to the site of attachment of the pH tester 238.

A third test procedure for detection and diagnosis of BV can be analysis for the presence of amines in the biological sample. In the embodiment of the device 210 shown in FIG. 1A, the distal end 240 of the test chamber 220 is adapted for storage and convenient, timely dispensing of an amine testing medium 204 used in the performance of an amine test as an indicator of BV. The distal end of the test chamber 240 is proportioned to can hold between about 0.1 ml to about 5 ml of solution. In one form of the invention, the test liquid comprises dilute aqueous alkaline liquid for reaction, as with bacteria in the vaginal moisture in the swab. One example of the alkaline test liquid is between about 1% to 20% v/v aqueous solution of potassium hydroxide. More preferably, the alkaline test liquid is between about 5% to 10% v/v aqueous solution of potassium hydroxide. In another preferred embodiment, the volume of the solution is about 0.1 cubic centimeters (cc³), more preferably about 0.5 cc³, 1 cc³, 1.5 cc³, 2 cc³, 3 cc³, 4 cc³, 5 cc³, 6 cc³ up to 20 cc³. When such solution contacts amines resulting from bacterial activity, a characteristic odor is produced, as by formation of gaseous amines such as cadaverine and/or putrescine. The user may thereby quickly and efficiently determine the existence of bacteria such as pathogenic bacteria in the vaginal moisture, using the test means and procedures described herein.

A preferred amine testing medium is a solution of about 10% KOH, preferably formulated in a viscous medium, for example in an agar or synthetic gel, to prevent accidental spillage. Preferably, the viscous medium remains in a liquid form and does not solidify at temperatures from about 20° C. to about 40° C. As used herein, “viscous medium” means a flowable material which has a viscosity substantially greater than water i.e. 5 times or more the viscosity of water. However, any amine testing medium able to facilitate evolution of trimethylamine or other amine vapors from the sample can be used for this purpose. When BV is present, an amine test solution such as KOH mixed with the sample will produce a noticeably fishy smell. In embodiments of the device 210 (FIG. 1A) utilizing KOH in the amine testing medium, the distal portion 240 or amine tester chamber, of the test chamber 220 can be tinted, for example a brown color, or constructed with light impermeable material which advantageously could also be an opaque material, or otherwise shielded from light, to prevent exposure of the KOH to light. However, KOH can be purchased from providers in plastic slightly opaque containers. Therefore, the amine test chamber can be constructed of plastics that vary in color and opacity such as white, slightly opaque through to a dark colored opaque plastic. The storage of KOH is well known to one of ordinary skill in the art. Examples of materials that are light impermeable or impervious include, but not limited to dark colored plastics, such as perspex or high density polypropylene or polytetrafluoroethylene material which may be colored black, rubber, metal. Alternatively the distal portion 240 or amine tester chamber of the test chamber 220 may comprise a coating which is highly internally reflective such as a silver or aluminum coating. Generally the coating will be a plastics coating. The coating should be sufficiently thick and dense and the material will have properties suitable for the application. Frequently the coating will be of a black plastics material.

In the device 210 shown in FIG. 1A, the amine testing medium 204 present in the distal portion 240 of the test chamber 220 is separated from the proximal portion 230 of the test chamber 220 by a breakable seal 235. The seal 235 can be fabricated from any suitable material capable of being broken, either by manipulation of the test chamber 220, or by piercing of the seal or septum 235. The seal or septum (i.e. a partition or dividing wall) serves as a self-closing inlet to prevent contamination. The seal 235 will open upon contact with the sample collecting tip 216, or other instrument used to introduce the vaginal fluid into the chamber 240, and will close or reseal upon removal of the collecting tip 216 or other such instrument. The septum is preferably constructed of a sealable material such as, for example elastomer, silicone rubber, teflon, etc. As used herein, the term “sealable” means that after introduction of sample, the septum will be able to close and maintain a closed or sealed environment without introduction of unwanted air, liquid, etc. from the outside and without substantial loss of air, fluid, etc. from the inside.

In a preferred embodiment, shown in FIG. 1A, in which the seal 235 is broken by manipulation of the distal portion 240 of the test chamber 220, the distal portion 240 can be fabricated from a deformable material such as flexible plastic or metal, for example of the types used in commercial toothpaste containers. The seal 235 can be fabricated of a material, such as metal foil or wax, capable of breaking upon deformation of the distal portion 240 of the test chamber 220. Upon deformation of the distal portion 240 of the test chamber, for example by squeezing, and breakage of the seal 235, the amine testing medium 204 can enter the proximal portion 230 of the test chamber 220 and can contact the biological sample on the collection tip 216.

In some instances it may be desirable to preserve a specimen of the biological sample, in addition to testing the sample using the device 210. For example, a sample may be preserved for subsequent diagnostic testing by a clinical facility, such as bacterial culture, or microscopic analysis, for example for the presence of clue cells (a diagnostic indicator of BV). In this case, the sample collection tip is not contacted by any hydroxide as this would lyse any cells in a sample. Rather a cell preservation medium is provided. Accordingly, as illustrated in FIG. 1D some embodiments of the device 210 include an optional sample preservation unit 270, including a container 275 proportioned to accommodate a sample collector 211 and a cap constructed to provide a leak-proof seal. The sample preservation unit 270 can include, within the container 275, a sample preserving medium 280, selected according to the intended diagnostic purpose. For example, to preserve cells in the sample for later microscopic analysis of clue cells, the biological sample on the tip 216 (FIG. 1A) or 218 (FIG. 1C) can be contacted with a conventional fixative solution 280 suitable for the purpose.

In practice, a biological sample is obtained as described above using a sample collector 211 (FIG. 1A), and the sample tip 216 or 218, with the sample disposed thereon, is brought into contact with the preserving medium 280 (FIG. 1D). In the embodiment illustrated in FIG. 1D, the handle 212 of the sample collector 211 fits the open end (not shown) of the container 275, forming a leak-proof seal. Following contact of the sample with the sample preservation medium 280, the sample is preserved for future testing, and contained in a leak-proof unit 270. Preferably a separate biological sample is used for performing the tests in the test chamber 220 and for preserving a sample in the sample preservation unit 270. The sample preservation unit 270 can include a label for recording information such as the subject's name and the date of acquisition of the sample or bar coded data that can be scanned into a computer readable format.

The kits of the invention can include instructions for 1) performing diagnostic tests for diagnosing BV, and 2) assigning a value to information derived from at least one determination made using the sample collector, pH tester and amine tester of the device 210. To detect the presence or absence of BV, the user performs a diagnostic test, and using the instructions, derives a numerical value based on one or more of the test results. Numerical results can be combined to arrive at a final numerical value for the test. By comparing the obtained numerical value to a predetermined control value of a non-BV infected subject, the user can predict whether or not the test results are in the range predictive of the presence of BV.

When using the devices and methods of the invention, the user performs the diagnostic test steps and assigns a numerical value to the obtained results, according to the instructions included with the kit. For example, the instructions may describe the steps to be taken in the performance of three tests, i.e., of the pH, appearance, and amine content of the biological sample. The instructions for interpreting the results of the tests (i.e., for assigning a numerical value to the results) can include the use of a scoring system, such as a score card 260 (FIG. 1E), for tabulating the results. Table 1 shows an exemplary scoring system for analysis of the three tests described above.

TABLE 1
Scoring System for Detection/Prediction of BV

| | Column 1 | Column 2 |
|---|---|---|
| | Put a 1 in the column | Put a 2 in the column |
| Characteristic | beside the correct finding | beside the correct finding |
| Color of pH paper | 4.5 or under | 5 or higher |
| after it is wet | | |
| from sample | | |
| Color and texture of | yellow, green, | white or gray |
| sample | clumpy white, | smooth or |
| | brown, red | creamy or frothy |
| Fishy smell when | No fishy odor | Fishy odor |
| sample is mixed | | |
| with amine test | | |
| solution | | |
| Scores | Column 1 | Column 2 |

Add the scores from Column 1 and 2 together. The Total Score is

In the practice of the invention, the user first obtains a sample of vaginal fluid or discharge from the subject (which may be herself). Any article suitable for obtaining the sample can be used. For example, a medically aseptic cotton-tipped applicator can be used as a sample collector. Using an embodiment such as the device 210 (FIG. 1A) however, the subject can conveniently grasp the holder 212 (FIG. 1A) of the collector 211 (FIG. 1A) and insert the shaft 214 with the collection tip 216 into the vagina, to collect a sample of vaginal fluid. The collector 211, with tip 216 moistened with the sample, is then placed in the proximal portion 230 of the test chamber 220 (FIG. 1A) for performance of the diagnostic tests.

Referring to Table 1, the subject is instructed to determine the pH of the sample, following contact of the sample with the pH tester 238 (FIG. 1A). In embodiments in which the pH tester 238 is a strip of pH paper attached to the test chamber 220 (FIG. 1A), the collection tip 216 is contacted against the strip of pH paper, so as to thoroughly wet the pH paper with the vaginal sample. In those embodiments in which the pH paper is incorporated into the collection tip 218 (FIG. 1C), this step is unnecessary, as wetting of the pH paper with the vaginal fluid occurs during sample collection. Alternatively, the invention can also be practiced by inserting a separate strip of pH paper (not shown) directly into the vagina of the subject, so as to wet the paper with vaginal fluid, then removing the pH paper for observation.

The user determines the pH of the sample by comparing the color of the pH paper after it is wet with vaginal fluid with the bands of color in the pH scale on the pH determining system 250 (FIG. 1B). Having determined a value for the pH (for example, 4.5), the user refers to the instructions and assigns a numerical value for the pH using a scoring system, such as a score card 260 (FIG. 1E). For example, as shown in Table 1, if the tested pH of the sample is 4.5 or lower, the user places the number 1 in Column 1 of the score card. If the pH of the sample is 5.0 or higher, the user places the number 2 in Column 2 of the score card 260. For each test characteristic, the user enters the appropriate number in either Column 1 or Column 2.

As another indicator of BV (i.e., a test predictive of BV), the user can observe the appearance of the vaginal sample and derive a numerical value from this information, according to the instructions. Referring again to Table 1, if the color of the sample seen on the collection tip 216 (FIG. 1A) or 218 (FIG. 1C) is yellow, green, clumpy white, brown or red, the user records the number 1 in Column 1. If the color of the sample is white or gray, with a texture that is smooth, creamy or frothy, the user places the number 2 in Column 2 of the score card 260 (FIG. 1E).

As yet another predictive test for BV, the user can determine the presence or absence of detectable amines in the vaginal sample. As describe above, an exemplary method for determining the presence of amines is the “whiff test,” in which a fishy odor can be detected when a vaginal sample from a subject with BV is contacted with an amine test solution, such as 10% KOH. Any suitable method can be used to perform the amine test. For example, a cotton-tipped applicator, or the collection tip 216 or 218 of the device 210 can be moistened with vaginal fluid or discharge, and then contacted with a KOH solution. Any suitable container can be used for contacting the sample with the KOH solution. A particularly preferred container for the KOH solution is an integrated container such as the distal portion 240 of the test chamber 220, shown in FIG. 1A. Using an embodiment of the invention such as the device 210, the user can conveniently and safely perform the amine test in the same device as the other tests, without the need to handle a caustic test solution, such as KOH, by breaking the seal 235 and causing contact of the collection tip 216 (FIG. 1A) or 218 (FIG. 1C) with the amine testing solution 204 (FIG. 1A).

Optionally, the amines may be detected by indicator. Any indicator that changes color upon exposure to amines, and preferably amines in a fluid specimen that would otherwise be acidic, may be used. Bromocresol green is one example, and may be used here as well as in the pH test. Other examples are bromophenol blue, bromocresol purple, bromochlorophenol blue, nitrazine yellow, and various other indicators.

Referring again to Table 1, following contact of the sample with the amine testing solution, the user smells the sample, to detect the presence of a fishy odor, which is diagnostic of the presence of amines in the biological sample. If no fishy odor is detected, the user records the number 1 in Column 1. If a fishy odor is detected, the user records the number 2 in Column 2.

Detection of a positive or negative diagnosis of BV is made by comparing numerical values derived from the tests of the sample with a predetermined value derived from test results from control subjects without BV, which is provided with the instructions for the device 210. As an example, using the criteria and scoring system set forth in Table 1, the user enters a value either in Column 1 or Column 2 for each of the three characteristics of the sample, then takes the sum of the numerical values recorded in the columns, to obtain a total score. A total score of 4 or higher is indicative of the presence of at least one diagnostic indicator of BV. In this example, the columns in Table 1 are arranged such that the characteristics listed in Column 1 (i.e., pH 4.5 or under, color and texture of discharge as listed, and absence of fishy smell) are all indicative of a negative diagnosis of BV. Conversely, the characteristics listed in Column 2 are all indicators of a positive diagnosis of BV. Accordingly, a score of 4, 5 or 6 is achieved when one, two and three positive diagnostic indicators of BV, respectively, are detected upon performing the tests. The instructions can include directions to seek the advice of a physician upon determination of a value above a predetermined normal value (the normal value being 3 in this example).

A particularly advantageous feature of the system is that it empowers a non-medically trained user to conveniently and comfortably screen herself (i.e., to perform a predictive self-diagnosis) or to screen others for the presence of BV infection at any time that concerns about such infection may arise. Having obtained a value that is predictive of the presence of BV, the user would be better informed as to the likely existence of an infectious condition, and would be directed to seek timely medical confirmation of the predicted diagnosis, and to obtain appropriate treatment. Accordingly, the invention can provide earlier diagnosis and treatment of BV, thus decreasing the risk of known deleterious effects associated with delayed treatment.

Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

All publications and patent documents cited in this application are incorporated by reference in their entirety for all purposes to the same extent as if each individual publication or patent document were so individually denoted. By their citation of various references in this document, Applicants do not admit any particular reference is “prior art” to their invention.

### EXAMPLES

In an ongoing study twenty subjects were informed of the procedure and consented to take part in the study. Nineteen subjects tested themselves and as the result of testing and scoring, visited the provider for diagnosis of the vaginal complaint. Two subjects returned twice (see below). 17 of the 19 subjects who thought they had BV from the scoring after self-testing were confirmed to have BV—these were initial checks. All 17 were treated with Metrogel successfully without any adverse events.

2 of the 19 thought they had some other vaginitis based on the testing and scoring, and in fact had candidiasis. They were treated by the provider for candidiasis. These 2 subjects (from different locations) returned at a later time after that infection had cleared with what they thought was BV and were confirmed to have BV by the provider. They were also treated with Metrogel without any adverse events.

Ten more study participants have been enrolled. Three of the participants scored positive for bacterial vaginosis and were confirmed to have bacterial vaginosis.

1. A system for detecting the presence of bacterial vaginosis comprising:
a sample collector for obtaining a biological sample from a subject;
a test chamber having an optically transparent portion to allow visual determination of an appearance of said sample while said sample collector is in said chamber;
a pH tester to indicate a pH of said biological sample; and
an amine tester.

2. The system of claim 1, further comprising a scoring system for tabulating a value, wherein a tabulated score above a control value is predictive of bacterial vaginosis.

3. The system of claim 1, further comprising a sample preservation unit.

4. The system of claim 1, wherein said pH tester is integrated into said test chamber.

5. The system of claim 1, wherein said pH tester is integrated into said sample collector.

6. The system of claim 1, further comprising a removable sample collector and a test chamber for inserting said sample collector therein.

7. The system of claim 1, wherein the sample collector comprises a sample holder, a shaft and a collection tip.

8. The system of claim 7, wherein the sample holder and the collection tip are affixed to the shaft at opposite ends of the shaft.

9. The system of claim 1, wherein the sample collector forms a leak-proof seal when inserted into the test chamber.

10. The system of claim 1, wherein the test chamber is constructed with an optically transparent material.

11. The system of claim 10, wherein the optically transparent material is polystyrene.

12. The system of claim 10, wherein the optically transparent material is glass.

13. The system of claim 7, wherein the collection tip comprises a white absorbent material.

14. The system of claim 1, wherein the sample collector is dimensionally proportioned for insertion into a human patient's vagina without discomfort.

15. The system of claim 1, wherein the test chamber further comprises an amine tester chamber separated from the collection tip by a breakable seal.

16. The system of claim 15, wherein the amine tester chamber protects the amine medium from light exposure.

17. The system of claim 15, wherein the amine tester chamber is constructed of opaque material.

18. The system of claim 1, wherein the amine tester is a hydroxide solution to evolve vapors from said sample for determination of a presence or absence of amines in said sample.

19. The system of claim 1, wherein the amine tester is a solution of about 10% potassium hydroxide formulated in a viscous medium.

20. The system of claim 1, wherein the amine tester is a solution of about 10% potassium hydroxide formulated in a viscous medium.

21. The system of claim 20, wherein the viscous medium is agar.

22. A system for detecting the presence of bacterial vaginosis comprising:
a sample collector for obtaining a biological sample from a subject;
a test chamber having an optically transparent portion to allow visual determination of an appearance of said sample while said sample collector is in said chamber;
a pH tester to indicate a pH of said biological sample; and,
a sample preservation unit.

23. The system of claim 22, wherein the sample preservation unit further comprises a container dimensionally proportioned to accommodate a sample collector and a cap.

24. The system of claim 23, wherein the cap forms a leak-proof seal when affixed to the sample preservation unit.

25. The system of claim 22, wherein the sample preservation unit contains a sample preserving medium.

26. The system of claim 22, wherein the sample collector comprising a biological sample contacts sample preservation medium contained in the sample preservation unit.

27. The system of claim 26, wherein the biological sample comprises cells from human vaginal fluid.

28. A method for detecting the presence of bacterial vaginosis, comprising the steps of:
(a) providing a biological sample from a subject;
(b) determining a value from at least one indicator of bacterial vaginosis;
(c) determining the sum of said at least one value;
(d) comparing the sum from step (c) to a control sum; and
(e) predicting the presence or absence of bacterial vaginosis based on said comparison.

29. The method of claim 28, wherein said indicator of bacterial vaginosis includes a visual bacterial vaginosis predictive value of said sample.

30. The method of claim 29, wherein a visual bacterial vaginosis-predictive value is correlated with a white or gray color and a smooth or frothy texture, and a visual non-bacterial vaginosis predictive value is correlated with a yellow, green, brown or red color and a clumpy texture.

31. The method of claim 26, wherein said indicator of bacterial vaginosis includes a pH value of said sample.

32. The method of claim 29, wherein a pH bacterial vaginosis-predictive value is correlated with a pH of 5.0 or higher, and a pH bacterial vaginosis-non-predictive value is correlated with a pH of 4.5 or lower.

33. The method of claim 26, wherein said indicator of bacterial vaginosis includes the presence or absence of amines in said sample.

34. The method of claim 28, wherein a bacterial vaginosis-predictive value is correlated with the presence of amines in said sample, and a bacterial vaginosis-non-predictive value is correlated with the absence of amines in said sample.

35. The method of claim 28, wherein said biological sample is vaginal fluid or discharge.

36. A kit for detecting the presence of bacterial vaginosis comprising:
a sample collector for obtaining a biological sample from a subject;
a test chamber having an optically transparent portion to allow visual determination of an appearance of said sample while said sample collector is in said chamber;
a pH tester to indicate a pH of said biological sample;
an amine tester to evolve vapors from said sample for determination of a presence or absence of amines in said sample; and
instructions for assigning a numerical value to at least one of said appearance, said pH and said presence or absence of amines, and comparing said value to a predetermined control value provided.

37. The kit of claim 36, wherein the visual determination of sample indicative of bacterial vaginosis is correlated with a white or gray color and a smooth or frothy texture, and a non-bacterial vaginosis sample is correlated with a yellow, green, brown or red color and a clumpy texture.

38. The kit of claim 36, wherein an indicator of bacterial vaginosis includes a pH of said sample.

39. The kit of claim 36, wherein a bacterial vaginosis-predictive pH value is correlated with a pH of 5.0 or higher, and a bacterial vaginosis-non-predictive value is correlated with a pH of 4.5 or lower.

40. The kit of claim 36, wherein a bacterial vaginosis-predictive value is correlated with the presence of amines in said sample, and a bacterial vaginosis-non-predictive value is correlated with the absence of amines in said sample.

41. The kit of claim 36, wherein the presence of amines in said sample are detectable by a fish-like odor in the presence of potassium hydroxide in a concentration of between about 5% to 20% v/v.

42. The kit of claim 36, wherein the presence of amines in said sample are detected by an amine indicator.

43. The kit of claim 42, wherein the amine indicator is selected from the group consisting of bromocresol green, bromophenol blue, bromocresol purple, bromochlorophenol blue, and nitrazine yellow.
